(19) United States
(12) Patent Application Publication
(10) Pub. No.: US 2019/0153386 A1
(43) Pub. Date: May 23, 2019

(54) STEM CELL LINE FOR TREATMENT OF VARIOUS MEDICAL CONDITIONS

(71) Applicants: James R. Musick, Conifer, CO (US); Tiana Tonrey, Wheat Ridge, CO (US); Richard Dean, Thornton, CO (US)

(72) Inventors: James R. Musick, Conifer, CO (US); Tiana Tonrey, Wheat Ridge, CO (US); Richard Dean, Thornton, CO (US)

(21) Appl. No.: 15/816,982

(22) Filed: Nov. 17, 2017

(51) Int. Cl. C12N 5/0797; C12N 5/0793; C12N 5/077; C12N 5/071; A61K 35/12

(52) U.S. Cl. CPC C12N 5/0623; C12N 5/0619; A61P 25/28; C12N 5/0692; A61K 35/12; C12N 5/0657

(57) ABSTRACT

An isolated biological stem cell or a set of biological stem cells that include human mesenchymal stem cell line 120816CT, deposited under ATCC Accession number PTA-124321, and differentiated into at least one type of cell selected from the group: neural stem cells, nephron progenitor cells, cardiomyocytes, neurons, glial cells, and vascular endothelial cells. The neurons comprise cholinergic neurons or dopaminergic neurons. A method of treating at least one human physical condition is also disclosed. The method includes injecting a patient with stem cell line 120816 CT to treat osteoarthritis, tendonitis, herniated disc, ligament damage, acute and chronic kidney disease and injury, Parkinson's disease, traumatic brain injury, Alzheimer's disease, amyotrophic lateral sclerosis, spinal cord injury, other skeletal muscular disorders, Lupus erythematosus, multiple sclerosis, cardiovascular disease, Graft-versus host disease, liver dysfunction or diseases, Type 1 and Type 2 diabetes, myocardial ischemia, heart failure, coronary artery disease, and other disorders characterized by inflammation.

### FIELD OF THE INVENTION

The present invention is related to treating various medical conditions by use of human mesenchymal stem cells (MSCs) and derivatives thereof.

### BACKGROUND OF THE INVENTION

Stem cell therapy has been used for some time now to treat various hematologic conditions including leukemia, and lymphoma using hematopoietic cells (HSCs). Other types of stem cells are being investigated for effectiveness in other conditions. Conditions suitable for treatment by human mesenchymal stem cells (MSCs) include skeletal muscular injuries and diseases, inflammatory conditions, autoimmune diseases, cardiovascular diseases, kidney failure, and various neurological conditions. MSCs are administered systemically or by injection into specific sites such as the intra-articular space of joints.

Adult stem cells (MSCs) are particularly relevant to the present invention. These cells were initially described by Arnold Caplan (Caplan, Al and Bruder, S P, Trends Mol Med 7: 259-264, 2001). MSCs may be derived from various tissues including bone marrow, adipose-tissue, dental pulp, umbilical cord, amniotic fluid and membranes, placenta, and other sources well-known to those skilled in the art. Comparative studies suggest differences in potency, differentiation capacity, growth rates and other cellular characteristics depending on the tissue source used to procure MSCs. This may be related to several factors, including medical status of the donor and environmental factors specific to the stem cell niche.

MSCs derived from bone marrow and adipose tissue have been subject to numerous clinical trials that provide preliminary evidence of safety and efficacy. A classic feature of MSCs is trilineage differentiation into adipocytes, chondrocytes, and osteoblasts, although multiple other cellular lineages may be derived from MSCs, including neural, kidney, and cardiac cells. Hence, MSCs have been extensively studied in skeletal muscular conditions such as osteoarthritis (OA). Several studies support safety and efficacy by intra-articular injections into knees, hips, and shoulder joints of OA patients. In addition, several other conditions may be treated by MSC transplants or transplants of progenitor cells derived from MSCs including stroke, myocardial infarct, and congestive heart failure.

### BRIEF DESCRIPTION OF THE DRAWINGS

A complete understanding of the present invention may be obtained by reference to the accompanying drawings, when considered in conjunction with the subsequent detailed description, in which:

FIG. 1 is a table showing a flow cytometry summary of all flow cytometry testing;

FIG. 2 is a set of graphs illustrating flow cytometry data for CD11b, CD14, and CD19;

FIG. 3 is a set of graphs illustrating flow cytometry data for CD34, CD44, and CD45;

FIG. 4 is a set of graphs illustrating flow cytometry data for CD73, CD79a, and CD90;

FIG. 5 is a set of graphs illustrating flow cytometry data for CD105, CD126, and HLA-DR;

FIG. 6 is a graph illustrating mesenchymal stem cell potency testing, the standard curve intracellular ATP, determined by a luciferin-luciferase method, shown on the left and the cellular ATP content shown on the right thereof;

FIG. 7 is a graph illustrating mesenchymal stem cell endotoxin testing, the standard curve on the left and sample data on the right thereof; and

FIG. 8 are images showing the result of mesenchymal stem cells adventitious agent testing, specifically PCR results of 16S, 18S, and mycoplasma testing.

Like reference numerals refer to like parts throughout the several views of the drawings.

### SUMMARY OF THE INVENTION

In accordance with the present invention, there is provided a method and compositions related to stem cell therapies. An isolated biological stem cell or a set of biological stem cells that include human mesenchymal stem cell line 120816CT, deposited under ATCC Accession number PTA-124321, and differentiated into at least one type of cell selected from the group: neural stem cells, nephron progenitor cells, cardiomyocytes, neurons, glial cells, and vascular endothelial cells. The neurons comprise cholinergic neurons or dopaminergic neurons. A method of treating at least one human physical condition is also disclosed. The condition includes osteoarthritis, tendonitis, herniated disc, ligament damage, acute and chronic kidney disease and injury, Parkinson's disease, traumatic brain injury, Alzheimer's disease, amyotrophic lateral sclerosis, spinal cord injury, other skeletal muscular disorders, Lupus erythematosus, multiple sclerosis, cardiovascular disease, Graft-versus host disease, liver dysfunction or diseases, Type 1 and Type 2 diabetes, myocardial ischemia, heart failure, coronary artery disease, and other disorders characterized by inflammation. The condition is treated by injecting a patient with biological stem cells comprising a human mesenchymal stem cell line 120816CT, deposited under ATCC Accession number PTA-124321, the injection being selected from the group: intravenous infusion, intra-articular injection, and intra-spinal-disc injection.

### DETAILED DESCRIPTION OF THE PREFERRED EMBODIMENT

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

Stem cells of the present invention are derived from a variety of tissues including adipose tissue, umbilical cord, placenta, amniotic fluid or membranes, umbilical cord blood, bone marrow, and other sources well known to those skilled in the art. The MSCs are extracted from tissue through a combination of microdissection followed by enzymatic or mechanical dissociation. Donor screening is used to eliminate those with pre-existing conditions that may impose a safety risk for use of stem cells in clinical applications. MSCs are purified by a variety of methods such as flow cytometry or selective adsorption. Expansion occurs by standard methods of cell culture, preferably performed under conditions of reduced oxygen (1% to 5%). Expansion of cells occurs by serial passage in cell culture using various methods of cellular dissociation from culture flasks/plates well known to those skilled in the art. MSCs are then characterized by a variety of methods to ensure authenticity by function including differentiation capacity, growth rate, ATP production, expression of indoleamine 2,3-dioxygenbase induced by γ-IFN and other MSC functional characteristics known to those skilled in the art. Also, phenotypic characterization is performed using flow cytometry to determine the absence or presence of specific cellular biomarkers. Karyotyping, DNA finger printing, and other well-known methods are used to authenticate the species of origin and elucidate the genome of the MSC line. Adventitious agents are determined by species-specific PCR methods or broader PCR methods detecting several different species within a genus. Detection of bacteria, fungi, and viruses is used to eliminate transmission of these agents during stem cell transplantation.

The MSC lines derived by the above methods are formulated by various methods including cryopreservation, viable liquid formulations that are suitable for administration into patients through various routes including both systemic and local applications. The MSCs may be genetically modified prior to clinical use.

The MSCs are distributed to various administration sites in suitable, stable formulations to accommodate logistic requirements including temperature maintenance, continuous monitoring of environmental parameters, etc. Stem cells are administered to a patient in need by various routes of administration, including, but not limited to, intravenous infusion, intra-articular injection, intra-spinal-disc injection, or other methods of systemic administration or local injection or implantation well known in the art, including combination with stabilizing agents such as extracellular materials, either natural or synthetic. Medical conditions suitable for the stem cell therapies embodied within the present invention include, without limitation, skeletal muscular conditions such as osteoarthritis, tendonitis, and ligament repair; anti-inflammatory applications, including cardiovascular risk reduction; auto-immune disorders such as Lupus erythematosus, and multiple sclerosis; liver dysfunction or diseases; kidney disease both acute and chronic; and various other disorders characterized by inflammation; neurological conditions including Alzheimer's disease, traumatic brain injury, concussion, Parkinson's disease, amyotrophic lateral sclerosis, dementia, Huntington's chorea, meningitis, stroke, autism spectrum disorders, memory loss, spinal cord injury, and impaired cognitive function.

The present invention utilizes MSCs preferably derived from human umbilical cord, placenta or amniotic tissues. Tissue MSCs are extracted from surgically dissected tissues using collagenase, protease or other means of cellular dissociation and subsequently purified by their attachment to polystyrene contained within standard cell culture flasks and plates. Conventional methods of cellular expansion are then used to expand attached MSCs. MSCs are characterized according to various methods to authenticate the cells by functional, structural, and genetic expression as well as determination of differentiation capability. Furthermore, numerous tests for adventitious agents are embodied by the present invention, including measurement of bacterial, fungal or viral infection. Genetic screening of MSCs is also embodied within the present invention.

The present invention also embodies various MSC treatments before and after transplantation to further enhance stem cell therapies. All treatments that enhance therapeutic benefit of the stem cell transplant are embodied within the present invention. A preferable method involves concomitant GSK3-beta inhibition with histone deacetylase inhibition either pre- or post-transplantation. Several methods of such pharmacologic therapy are well known in the art, including lithium and valproic acid (Depakote) or nutraceuticals including curcumin and quercetin, as disclosed in U.S. patent application Ser. Nos. 15/340,117 and 15/340,125. Stem cell status may be monitored by measuring serum levels of CXCR4, SDF1-alpha, PGE2, and MMP9.

The mechanisms by which stem cells exert therapy are not yet completely understood, but substances secreted by stem cells have various paracrine biological effects including trophic effects, growth effects, neural protection, immunological modulation, anti-microbial effects, and anti-inflammatory effects. Also, exosomes are intracellular vesicles secreted by stem cells that also exert therapeutic benefit. Hence, the present invention also embodies the secretome of MSCs including all soluble and membrane bound biological agents. Some therapies may be elicited by use of cell-free formulations of MSCs embodied herein. Also, stem cells are well known to differentiate into a wide variety of different lineages and to exert cellular effects through intercellular communication via connection through tunneling nanotubes.

The present invention also embodies derivatives of MSC lines including differentiated cell lineages both progenitor cell lines, including but not limited to, neural stem cells (NSCs), nephron progenitor cells (NPCs), and terminally differentiated cells as well. NSCs may be differentiated into neurons, glia cells or vascular endothelial cells including dopaminergic and cholinergic neurons by methods well-known in the art. Such terminally differentiated cells may be used therapeutically to treat Parkinson's disease and amyotrophic lateral sclerosis (ALS) disease. Likewise, other disease conditions may benefit from transplantation of terminally differentiated cells derived from MSC lines either alone or in combination with non-differentiated MSCs, PRP, etc.

### EXAMPLES

Extraction of Mononuclear Cells from Human Umbilical Cord Methods: Human umbilical cord was obtained from CryoPoint, Brownsville, Ind. under informed consent from the donor. It was transported in saline and maintained at 4° C. during transport. The umbilical cord was micro-dissected into 1-2 cm² pieces in a petri dish containing phosphate buffered saline (PBS) within a biological safety cabinet. All subsequent procedures occurred in a sterile environment. An enzymatic digestion mixture was prepared in 0.2 PZ U/ml Collagenase NB 6 GMP grade, Serva Chemicals, in HEPES-buffered saline at 2.5 ml/gm umbilical cord tissue. This mixture was incubated at 37° C. in an end-over-end rotator at 25 RPM. Following incubation, cells and tissue were separated using a Buchner funnel (90 to 130-micron pore size) following washout of residual cells, with 3×20ml PBS. Cells were pelleted by centrifugation at 440×G for 15 minutes and re-suspended in 10-20 ml PBS for cell counting including total cell count (10 to 40 micron) and acridine-orange, propidium iodide using the Countess II instrument (Fisher Scientific). The mononuclear cells were re-suspended at 10⁶ MNC/ml in CryoStore 2 cryopreservation medium (BioLife Solutions, Catalog Number 202102) and cryopreserved using controlled rate freezing from room temperature to −80° C. at −1° C./minute, followed by storage in liquid nitrogen.

Results: The above procedures yielded a total cell count of 530 million, consisting of 13 million MNCs at 47.3% viability.

Expansion of MSCs

Methods: Pass 0 cultures, i.e., initial passage following collagenase digest, were cultured at 15K to 20K total cells/cm² in T-75 TC-coated tissue culture flasks (Falcon, Catalog Number 353136) in MSC cell culture medium (MSC-Gro™, Vitro Biopharma catalog number SC00B4-3, Clinical Grade Humanized, Serum-free medium, (supplemented to 5% Human serum AB, Golden West Biologicals, Temecula, Calif.) plus lx penicillin/streptomycin (Sigma, catalog number P4383). The cultures were maintained in a tri-gas incubator (HerraCell, 240i; Fisher Scientific) set to 5% O₂ and 5% CO₂. These cultures were monitored at 4 to 5-day intervals for colony formation and expansion. Pass 0 cultures were maintained for 10 to 14 days with feeding at every 3 days following establishment of cultures at about 7 days. At 80% to 90% confluence, the Pass 0 cultures were sub-cultured using TrypLE™ Select (1×) (Gibco, Catalog Number 12563-029) using 30-minute incubation at 37° C. with agitation at 75 RPM. Similar procedures were used in Passes 1, 2, and 3, except that T-1000 flasks were used for these passages and appropriate scale-up in media volumes used for culture, TrypLE™ Select, etc.

Results: A single T-75 culture of pass 0 cells yielded on average 8 to 10 million MSCs at >85% viability. Subsequent passages yielded 10 to 12-fold increases in the number of inoculated MSCs at greater than 90% viability.

Characterization of MSCs Derived from Human Umbilical Cord

Methods: Flow cytometry utilized the following PE-conjugated antibodies at the indicated dilutions.

| | Miltenyi | | |
|---|---|---|---|
| | Biotech | | |
| | Catalog | | |
| ANTIGEN | Number | Dilution | MSCs |
| CD14 | 130-098-067 | 1:33 | 100 k/ml-1.5% |
| | | | HSA/PBS |
| CD19 | 130-098-068 | 1:33 | 100 k/ml-1.5% |
| | | | HSA/PBS |
| CD34 | 130-098-140 | 1:33 | 100 k/ml-1.5% |
| | | | HSA/PBS |
| CD44 | 130-098-108 | 1:100 | 100 k/ml-1.5% |
| | | | HSA/PBS |
| CD45 | 130-096-141 | 1:33 | 100 k/ml-1.5% |
| | | | HSA/PBS |
| CD73 | 130-097-943 | 1:100 | 100 k/ml-1.5% |
| | | | HSA/PBS |
| CD90 | 130-097-932 | 1:33 | 100 k/ml-1.5% |
| | | | HSA/PBS |
| CD105 | 130-098-906 | 1:33 | 100 k/ml-1.5% |
| | | | HSA/PBS |
| HLA-DR | 130-098-177 | 1:33 | 100 k/ml-1.5% |
| | | | HSA/PBS |

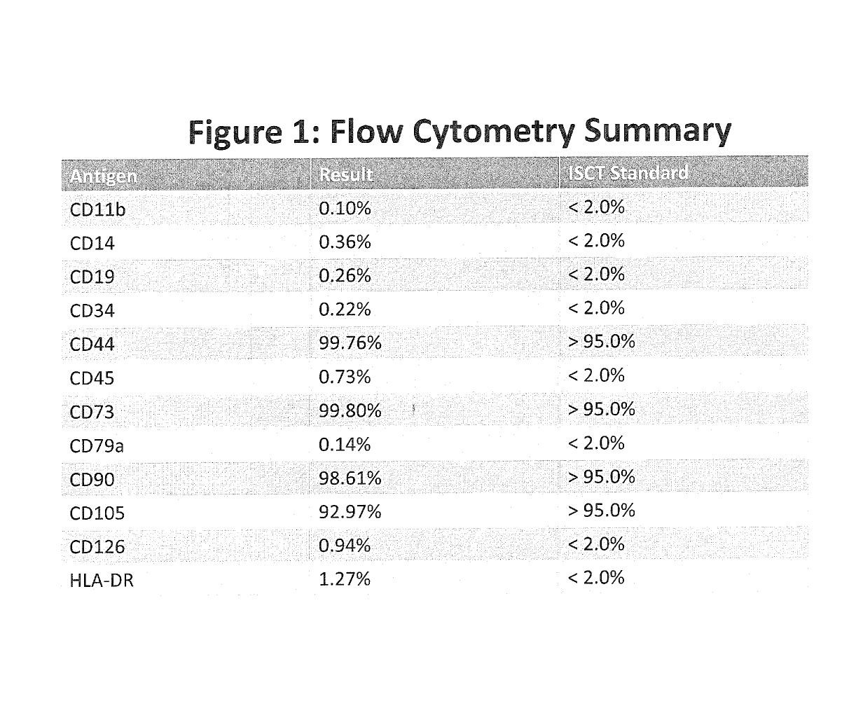

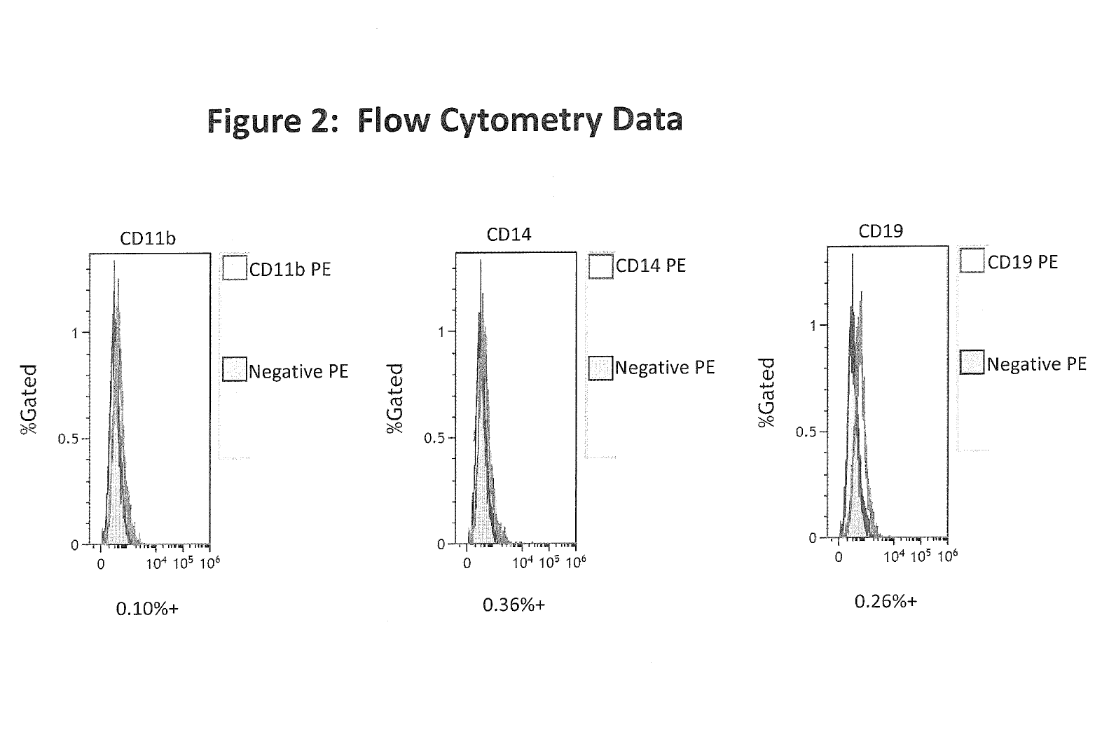

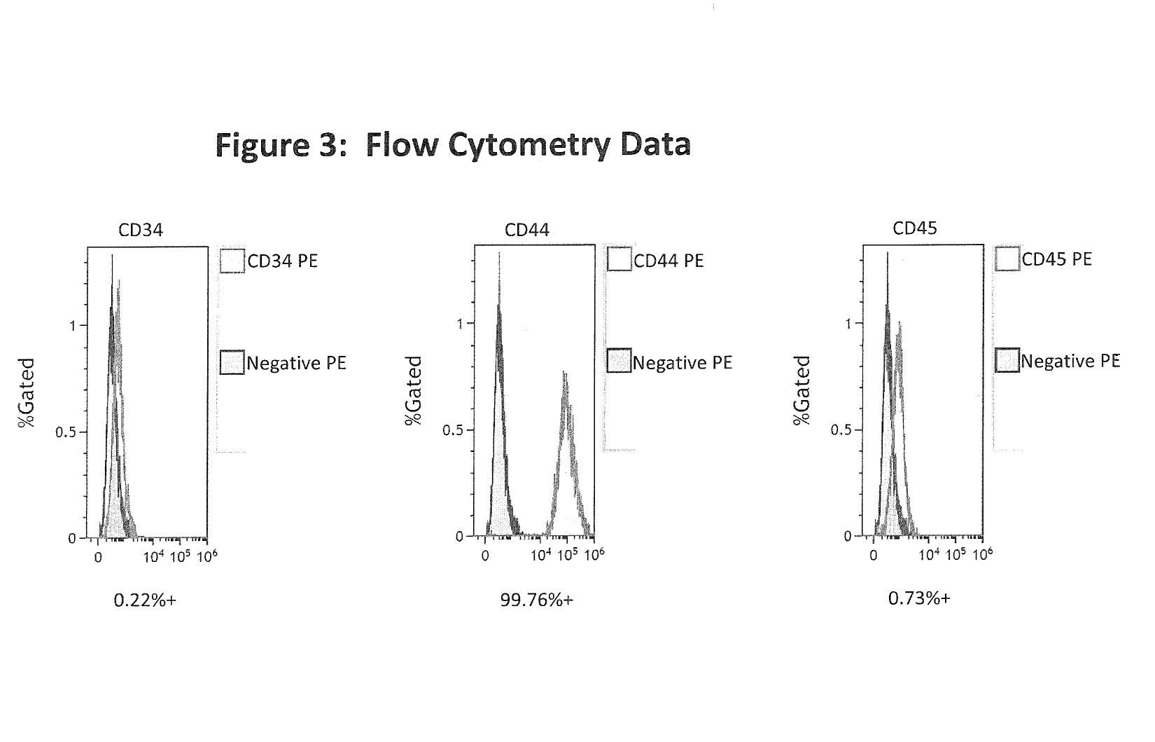

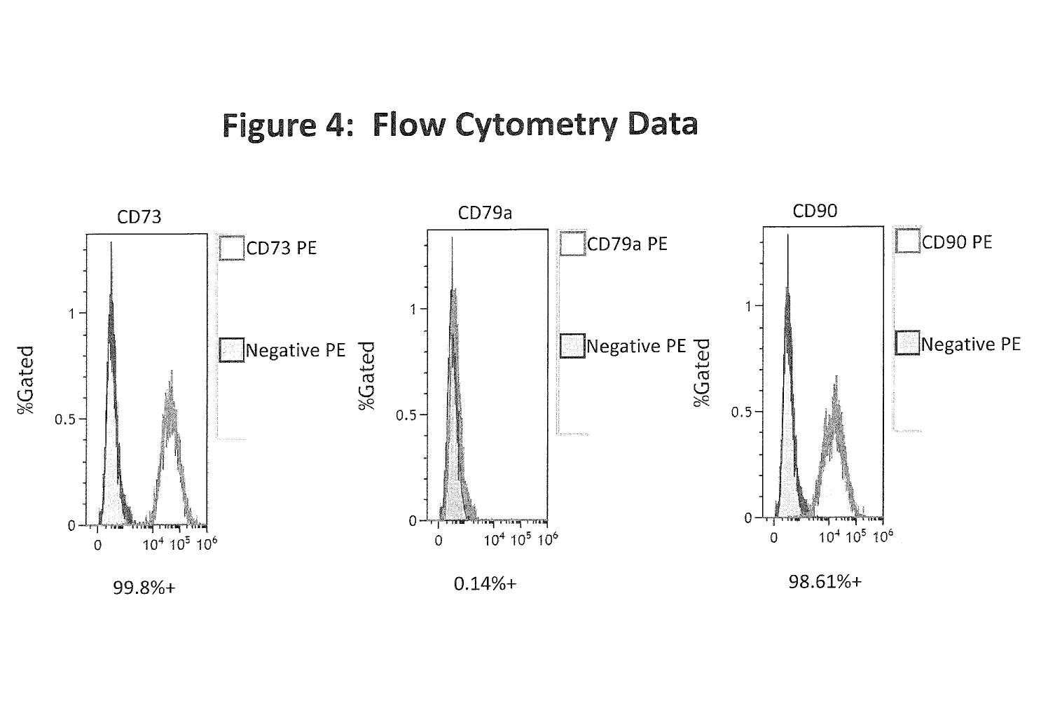

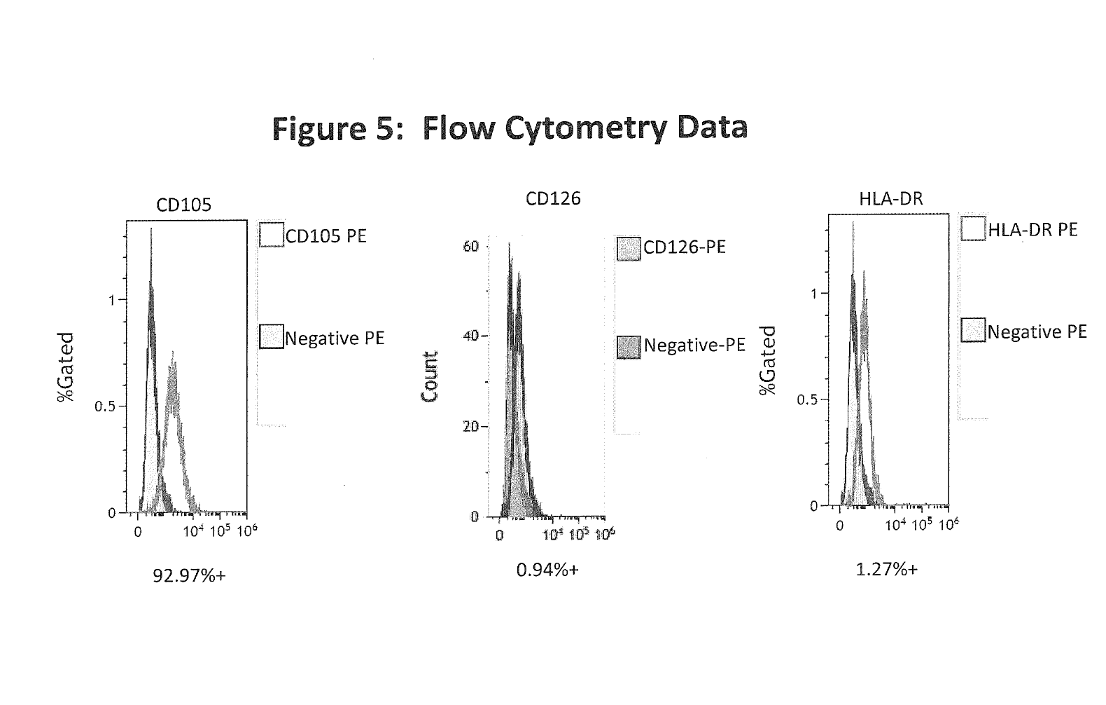

These mixtures were incubated for 1 hour at room temperature, followed by centrifugation at 400 g for 5 minutes. The cells were washed with 1 ml PBS and suspended in 1 ml PBS. Flow cytometry data was collected on a Gallios instrument (UC Denver Flow Cytometry Core Lab). A summary of the results is provided in FIG. 1 and histograms of the results are shown in FIGS. 2-5.

ATP cellular content was determined by intracellular ATP measurement using the luciferase-luciferin assay (Cell Titer Glo, Promega, catalog number G9681) according to the manufacturer's procedures. Luminescence was converted to [ATP] using an internal ATP standard curve (0, 0.25, 0.5, 1, 2, 4, 8, and 10 μM). Luminescence was read on a Turner Modulus microtiter plate reader.

PCR was used to determine contamination by mycoplasma, bacteria (16S ribosomal RNA) or fungi (18S ribosomal RNA). Genomic DNA was extracted from MSCs using a Qiagen DNeasy Blood and Tissue kit according to the manufacture's procedure. Bacterial contamination was determined by use of the Fast MicroSeq 500 16S rDNA bacterial identification kit (Applied Biosystems, catalog number 4370653) according to the manufacture's procedures. Fungal contamination was determined using MicroSeq D2 LSU rDNA Fungal Identification kit (Applied Biosystems, catalog number 4349160) based on determination of select sequences within 18S ribosomal RNA. The procedures used were those of the manufacturer. Mycoplasma was determined by use of the e-Myco Plus Mycoplasma PCR kit (iNtRON, catalog number 25234) according to the manufacturer's procedures. Endotoxin was determined by the Thermo Scientific Pierce LAL Chromogenic Endotoxin Quantitation kit (catalog number 88282) using the manufacturer's procedure.

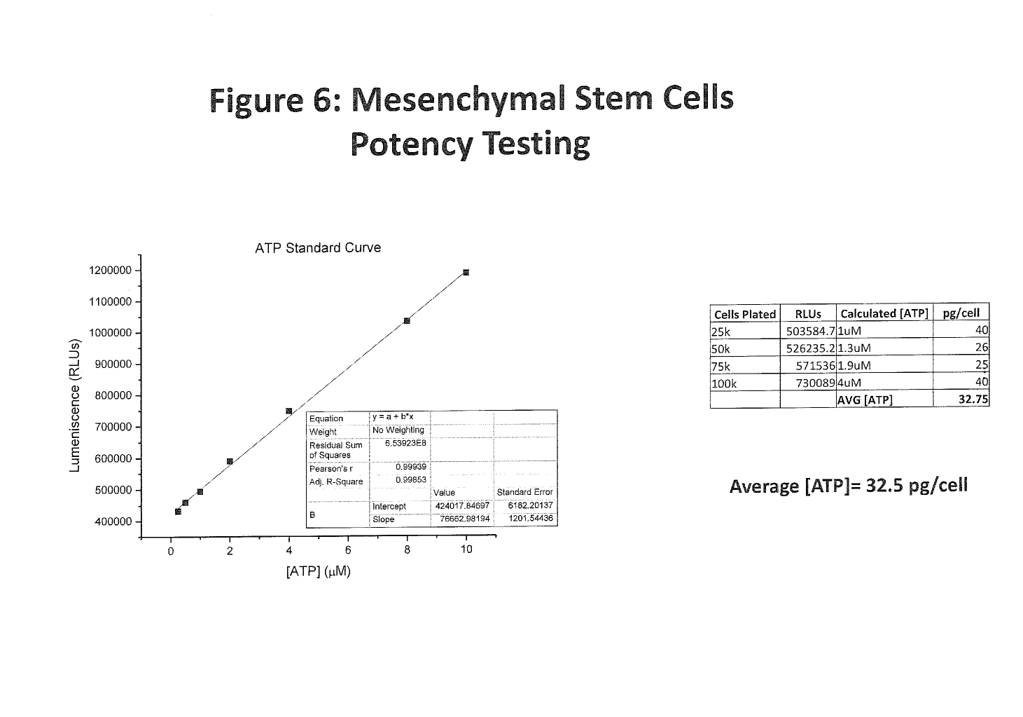

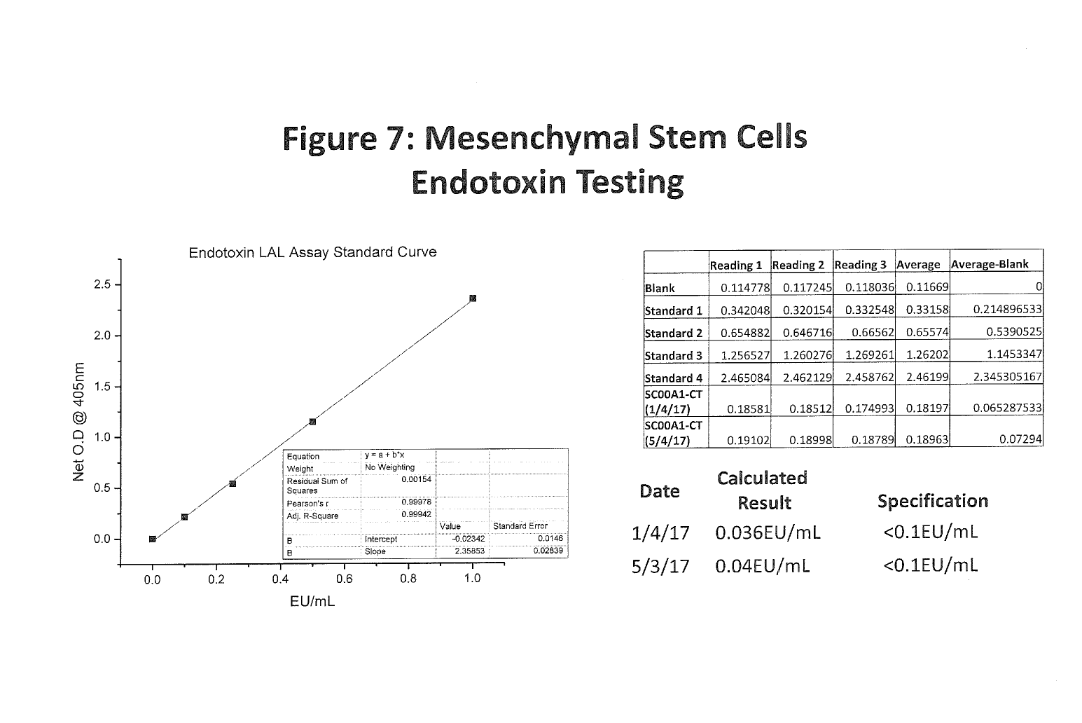

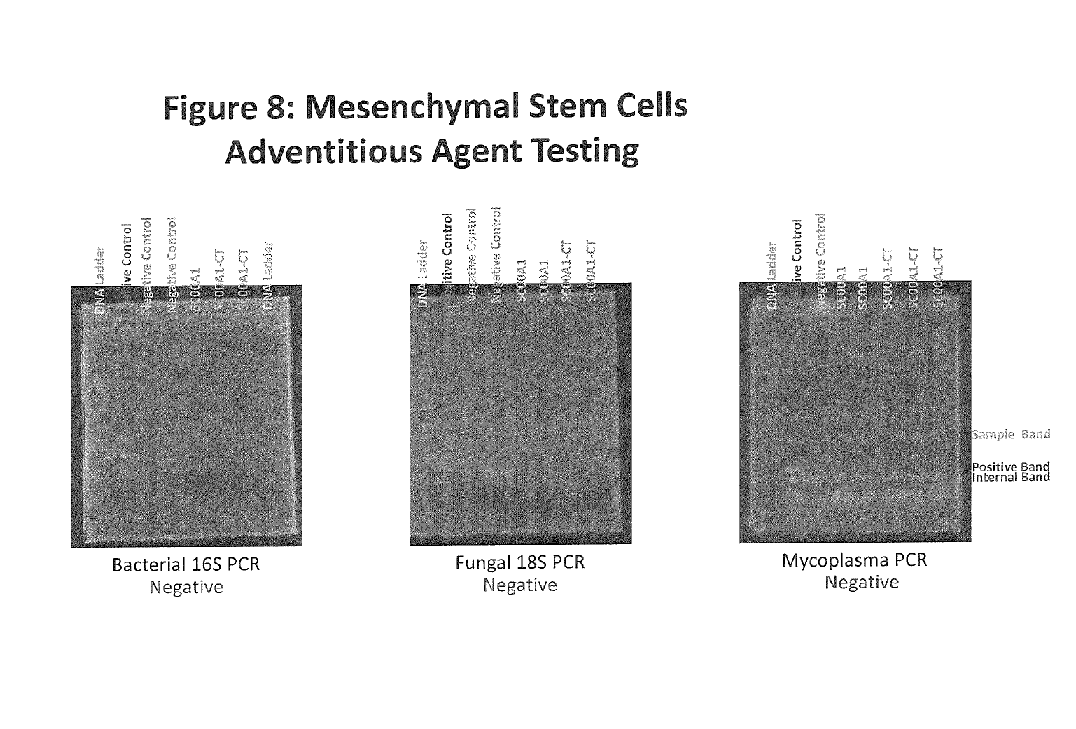

Results: Flow cytometry showed coincidence with ISCT standards for MSCs, with the exception of CD105 that was less than 95%. Potency testing showed an average ATP content per cell of 32.5 pg (FIG. 6). Endotoxin testing showed <0.1 EU/ml (FIG. 7). The cells were negative for bacteria, fungi, and mycoplasma by PCR methods (FIG. 8).

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

1. An isolated biological stem cell comprising a human mesenchymal stem cell line 120816CT, deposited under ATCC Accession number PTA-124321.

2. A set of biological stem cells comprising a human mesenchymal stem cell line 120816CT, deposited under ATCC Accession number PTA-124321, and differentiated into at least one type of cell selected from the group: neural stem cells, nephron progenitor cells, cardiomyocytes, neurons, glial cells, and vascular endothelial cells.

3. The set of biological stem cells of claim 2, wherein the neurons comprise at least one type of cell selected from the group: cholinergic neurons and dopaminergic neurons.

4. A method of treating at least one human physical condition selected from the group: osteoarthritis, tendonitis, herniated disc, ligament damage, acute and chronic kidney disease and injury, Parkinson's disease, traumatic brain injury, Alzheimer's disease, amyotrophic lateral sclerosis, spinal cord injury, other skeletal muscular disorders, Lupus erythematosus, multiple sclerosis, cardiovascular disease, Graft-versus host disease, liver dysfunction or diseases, Type 1 diabetes, Type 2 diabetes, myocardial ischemia, heart failure, coronary artery disease, and other disorders characterized by inflammation, the steps comprising injecting a patient with biological stem cells comprising a human mesenchymal stem cell line 120816CT, deposited under ATCC Accession number PTA-124321, the injection being selected from the group: intravenous infusion, intra-articular injection, and intra-spinal-disc injection.

5. The method of treating the human physical condition in accordance with claim 4, wherein an extracellular component of said human mesenchymal stem cell line 120816CT is injected into the patient, the injection being selected from the group:
intravenous infusion, intra-articular injection, and intra-spinal-disc injection.

6. The method of treating the human physical condition in accordance with claim 4, wherein the human mesenchymal stem cell line 120816CT is initially differentiated into neural stem cells, nephron progenitor cells or cardiomyocytes prior to clinical administration to a patient in need.

7. The method of treating the human physical condition in accordance with claim 4, wherein the human mesenchymal stem cell line 120816CT is initially differentiated into cholinergic or dopaminergic neurons prior to clinical administration to a patient in need.
